(19) United States
(12) Patent Application Publication
(10) Pub. No.: US 2005/0196373 A1
(43) Pub. Date: Sep. 8, 2005

(54) Cactus fruit skin care products

(76) Inventor: Jau-Fei Chen, Orem, UT (US)

Correspondence Address: Brent T. Winder;Jones Waldo Holbrook & McDonough, Suite 1500, 170 South Main Street, Salt Lake City, UT, 84101-1644, US

(21) Appl. No.: 11/007,945

(22) Filed: Dec. 9, 2004

Related U.S. Application Data
(62) Division of application No. 09/298,245, filed on Apr. 23, 1999, now abandoned.

(51) Int. Cl. A61K 7/06; A61K 35/78

(52) U.S. Cl. 424/74; 424/767; 424/728

(57) ABSTRACT

The present invention comprises a novel combination of cactus fruit extracts and other skin nutrients and moisturizers which may be used to soften and moisturize the skin while providing essential vitamins and nutrients to the skin in a natural way.

### RELATED APPLICATIONS

The following applications are being filed concurrently herewith on this 23^{rd} day of April 1999 and are incorporated herein by reference:

| Title | Atty Docket No. | Express Mailing Label Nos. |
|---|---|---|
| Ginseng Berry Topical | 7537.0027 | EL 113 362 505 US |
| Applications | | |
| Cactus Fruit Drinks and | 7537.0026 | EL 113 362 479 US |
| Food Products | | |
| Ginseng Berry Drink and | 7537.0028 | EL 113 362 482 US |
| Food Compositions | | |
| Ginseng Berry Powder | 7537.0030 | EL 113 362 496 US |
| Dietary Supplements | | |
| Cactus Fruit Powder | 7537.0031 | EL 113 362 465 US |
| Dietary Supplements | | |

### FIELD OF THE INVENTION

The present invention relates generally to the field of skin care products and more particularly to products and methods which deliver fresh vitamins and other nutrients to the skin by topical application of a novel, vitamin-rich fruit composition. The present invention comprises a novel combination of cactus fruit extracts and other skin nutrients and moisturizers which are beneficial to the skin.

### BACKGROUND

Human skin is extremely susceptible to the temperature and humidity extremes of our environment. However, when skin care products are properly used to counteract adverse environmental conditions, skin can remain healthy and beautiful under a variety of extreme environmental conditions. The environmental factors that most often affect the skin adversely are ultraviolet radiation and humidity.

Ultraviolet radiation varies with time of day, from day to night, with seasons of the year and weather conditions. The geographic region where one lives and the climate will also affect the amount of radiation to which one's skin is exposed. The sun's rays can dry skin through direct moisture loss or through the effects of radiation on the skin which may cause tanning and burning as well as moisture loss.

Skin may also face adverse conditions in the workplace where excessive temperatures or low humidity may harm skin. Exposure to chemicals may also remove moisture from the skin causing damage and actual skin chafing and loss if not treated properly.

In addition to environmental factors, skin must also be properly nourished. Maintaining healthy skin requires maintenance of proper moisture in the skin as well as delivery of essential vitamins to the skin. Vitamins may be consumed in the diet or may be applied directly to the skin.

For some people, oral consumption of vitamin C, especially in large doses, can have detrimental side effects ranging from mouth irritation to overdose. Yet large doses are sometimes necessary to provide the skin with the maximum beneficial amount of vitamin C. Vitamin C promotes collagen synthesis through its free radical scavenging attributes and its enzyme reactions which, in turn, promotes wound healing and skin health. Vitamin C is also toxic to many cancer cells including melanoma and has been found to catalyze the immune reaction to viral and bacterial infections.

Natural skin care products and remedies are popular among health-conscious consumers today. Many people prefer to enhance their appearance and health with vitamins and other nutrients in a “natural” way from naturally occurring sources. “Natural” products including natural vitamins are now in high demand. These are vitamins which are found in a product in its natural state without vitamin supplements or vitamin “fortification.” While fruit and vegetable juices are known to have high concentrations of vitamins in their natural state and are often a preferred source of vitamins for internal consumption, many natural fruit and vegetable products are largely overlooked as a topical skin application.

What is needed is a skin care product and method that delivers natural vitamins, nutrients and other beneficial products to the skin without oral consumption and its adverse side-effects. Also needed is a natural product which can moisturize as well as nourish the skin.

### SUMMARY AND OBJECTS OF THE INVENTION

Accordingly, it is an object of the present invention to provide a product and method which can deliver natural vitamins, skin nutrients and skin protectants to the skin in a topical application that nourishes and moisturizes the skin naturally.

The present invention comprises novel compositions of extracts from cactus fruit, herbs and other skin care ingredients which are mixed to form a topical application. A method of using these compositions is also a part of the present invention.

It is an object of preferred embodiments of the present invention to deliver natural vitamins to the skin.

It is another object of preferred embodiments of the present invention to deliver natural fruit and vegetable extracts to the skin so that the skin may benefit from natural vitamins, emollients and other healthful ingredients.

It is yet another object of preferred embodiments of the present invention to deliver natural and healthful herbs to the skin.

It is a further object of preferred embodiments of the present invention to moisturize the skin.

It is a still further object of preferred embodiments of the present invention to provide cactus fruit extract as a skin moisturizer and vitamin delivering agent.

### DETAILED DESCRIPTION OF THE PREFERRED EMBODIMENTS

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

The novel methods and compositions of the present invention allows users to apply natural vitamins, anti-oxidants and emollients directly to the skin and hair.

A preferred embodiment of the present invention comprises a fruit juice composition containing juice from cactus fruit. A preferred genus of cactus is the Cereus genus with several preferable species such as Cereus grandiflorus, Cereus giganteus and Cereus thurberi. Another preferred genus is the Opuntia which includes many preferred species including Opuntia strigil, Opuntia basilaris, Opuntia rufida, Opuntia phaeacantha, Opuntia engelmannii, Opuntia erinacea, Opuntia humifusa, Opuntia phaecantha, Opuntia chlorotica, Opuntia polycantha, Opuntia violacea, Opuntia spinosbacca, Opuntia lindheimeri and Opuntia macrorhiza. The species within the Opuntia genus of cactus have relatively flat, oval-shaped pads, similar to the leaves or branches of a bush, which may or may not have spines thereon. Cactus fruit or “pears” generally grow around the perimeter of the oval-shaped pads on these cacti. In the practice of the present invention, juice is derived from the cactus fruit. In some presently preferred embodiments, juice may also be derived from the cactus pads or stems as noted.

Cactus fruit grow in several varieties with varying color, seed content, sugar content and size. Colors range from green and yellow to purple, orange and red. Fruits generally range in size from 110 grams to 150 grams. Seed content, by weight, typically ranges between 2 and 4 grams per fruit while sugar content generally ranges between 11% and 16%. Cactus fruit used in one presently preferred embodiment of the process and compositions of the present invention are red with average to high sugar content from the Cereus Grandiflorus species.

Laboratory analysis of the juice from cactus fruit used for the preferred embodiment of the composition of the present invention shows a high concentration of essential vitamins. The following Table 1 gives the result of a laboratory analysis of cactus juice squeezed from fruit from the Cereus Grandiflorus species of cactus which is used in a preferred embodiment of the present invention.

TABLE 1

| | | |
|---|---|---|
| Thiamin | 116.6 | ug/gram of product |
| Riboflavin | 221.9 | ug/gram of product |
| Vitamin C | 8.35 | mg/gram of product |
| Vitamin A | 309 | IU |
| Vitamin E | 1.36 | IU |
| Vitamin D3 | 120 | IU |
| Beta Carotene | 16.6 | IU |
| Cyanocobalamine B12 | 91 | mg/gram of product |

Cactus fruit juice has also been found to be an effective anti-oxidant. Laboratory analysis reveals that one gram of cactus fruit contains 7 times more antioxidant than 10 mg of Vitamin C. The results of these tests prove that cactus fruit juice is an excellent source of vitamins.

As well as supplying nutrients beneficial to the skin and body, cactus fruit juice is also an effective skin moisturizer. Laboratory tests using skin surface hydration measurements show that cactus fruit extract has noticeable and enduring moisturizing effect on the skin. In these laboratory tests a tuberculin syringe was used to deliver 0.05 cc of cactus fruit extract to a 5×5 cm square on the volar forearms of 8 healthy volunteers. A glass rod was used to evenly spread the material over the test site. Immediately before treatment and at 2 and 4 hours thereafter, a series of skin surface hydration measurements were taken using a Skicon-200 conductance meter which was equipped with a Measurement Technologies probe. As a follow-up test the test sites were briefly wetted with water and the resulting change in conductance measured 2 minutes later. The results are tabulated below in table 2.

TABLE 2

| | No Treatment | Cactus Fruit Extract |
|---|---|---|
| Time | Mean ± SD | Mean ± SD |
| Baseline | 160.6 ± 48.2 | 163.3 ± 48.3 |
| Post 2 hours | 152.8 ± 52.6 | 203.2 ± 53.3 |
| Post 4 hours | 157.2 ± 51.2 | 201.8 ± 57.7 |
| 2 min. post wet | 292.4 ± 149.4 | 427.2 ± 164.8 |

Clearly, the skin surface hydration levels are markedly enhanced at the location where cactus fruit extract has been applied. These tests also show an enduring moisturizing effect lasting over 4 hours. Results of the follow-up test, shown in the bottom row of table 2, also show a significant increase in skin surface hydration levels after wetting. This indicates that the water binding capacity or the water retention properties have been noticeably improved by the application of cactus fruit extract leading to improved re-moisturizing as well as improved moisture retention.

Cactus fruit extract has also been found to promote proliferation of normal human fibroblasts thereby promoting wound healing. The Thymidine Incorporation Assay is commonly used to study proliferation of cells exposed to various chemicals. As cells grow, they must replicate their genome with every division cycle. If radiolabeled thymidine is present in the media, the cell will import this and incorporate it into the replicating DNA. Consequently, the rate at which cells divide can be quantified by the relative amounts of labeled thymidine in the cell. The goal of this study is to determine whether the addition of Cactus fruit extract to normal and transformed cells will increase the cell proliferation.

In this study, crude cactus fruit (21.17 g) was diluted in 25 ml of sterile Hanks buffer solution. The mixture was centrifuged for 5 min @ 1460 rpm to pellet debris. The supernatant was filtered through a 0.2-μm sterile filter.

Raji cells (established, transformed human lymphocytes) and FS-10 cells (normal human foreskin fibroblasts) were grown to log phase in RPMI media. 100 μl of cell suspension was added to cach of 5 wells on a 96-well plate for each dilution. Dilutions of cactus fruit extract were made in sterile Hank's buffer solution. 100 μl of each dilution was added to each well. Pure Hank's buffer was used as a control. Cells were incubated for 21 hours. H³-radiolabeled thymidine was added to RPMI media at a concentration of 1-μl thymidine (15.4 Ci/mmol): 49 μl media. 50-μl of thymidine solution was added to each well, and the plate was incubated for 3 hours. After 24 hours total incubation, cells were harvested using a cell harvester. The plates were allowed to dry overnight. Radioabsorption was subsequently measured.

The addition of cactus fruit extract to Raji cells (a cancerous cell line) did not cause a significant increase in thymidine incorporation. In fact, the levels were consistently lower than controls. Normal fibroblasts grown in the presence of cactus berry extract, however, showed as high as 38.6% increase in thymidine incorporation. Repeat experiments were performed with similar results. Only the results of FS-10 normal cells have been included in this document.

This preliminary study gives the indication that cactus fruit extract does promote cell proliferation in normal human fibroblasts but not in transformed cells. This could play a major role in wound healing. The extent to which cactus fruit extract promotes wound healing may be determined by further analysis. Regardless of the extent of this proliferating effect, this increase in cell proliferation may aid in healing of skin wounds caused by abrasions, contusions or lacerations as well as sores caused by acne or disease. Results of this analysis are shown in table 3 below.

TABLE 3
Cactus Fruit Extract
FS-10 normal human fibroblasts

| Dilution Factor | Absorption | % Increase |
|---|---|---|
| 1:4 | 1031.4 | 38.6% |
| 1:9 | 961.6 | 29.3% |
| 1:49 | 978.7 | 31.6% |
| 1:99 | 914 | 22.9% |
| 1:299 | 749 | 0.7% |
| Control | 743.9 | |

Specific embodiments of the present invention also contain a composition containing juice from the ginseng berry. Although the ginseng root is sometimes used as an herbal supplement, the ginseng berry has been overlooked due, partially, to its small size and high seed content. Ginseng berries contain a large number of seeds which make up a large percentage of the berry's volume. These seeds must be painstakingly removed in order to make a liquid suitable for topical skin applications.

Laboratory analysis of the juice from ginseng berries used for preferred embodiments of the composition of the present invention also show a high concentration of essential vitamins. The following Table 4 gives the results of a laboratory analysis of the essential vitamins and ingredients found in ginseng berry juice.

TABLE 4

| | | |
|---|---|---|
| Riboflavin | 171.9 | ug/gram of product |
| Vitamin C | No | |
| Vitamin A | 109 | IU |
| Vitamin E | 1.5 | IU |
| Vitamin D3 | No | |
| Beta Carotene | 16.9 | IU |

Advantageously, ginseng berry juice also acts as an anti-oxidant. Laboratory analysis reveals that one gram of ginseng berry contains 1.4 times more antioxidant that 10 mg of Vitamin C.

Modem machinery may be used to produce juice from the ginseng berry, cactus fruit and cactus pads, however, a preferred method of the present invention comprises a manual juice extraction process. The skin of the cactus fruit is peeled off by hand, using a knife, leaving the soft meat of the fruit exposed. Seeds are then removed by filtration through a coarse screen. The remaining meat is squeezed in a press to remove as much juice as can be practically removed. The squeezed meat is subsequently blended, preferably with a mixer-type appliance and filtered through a 0.2 micron filter using a micro-filtration system. Cactus pads are processed in a similar way.

Juice is extracted from ginseng berries in a similar way as well. Whole ginseng berries are squeezed in a press creating a somewhat pulpy extract. Seeds are then removed with a coarse screen filter. When a liquid is desired, the extract may be further filtered using a 0.2 micron micro-filtration system to remove solids. When a acceptable or desired, the micro-filtration may be omitted to retain pulp and solids. Juice may be extracted from ginseng berries, cactus pads and cactus fruit separately so that the two juices may be blended in specific proportions.

After juice has been extracted, it is blended with other natural ingredients which add other nutritional and physiological benefits.

Application of natural herb products along with the beneficial vitamins contained in cactus fruit juice may also increase health and vitality. The effects of various herbs and plant products are beneficial to the nervous, digestive and circulatory systems as well as other physiological functions. Herbs which, when applied to the skin, are beneficial to one's health and vitality may be considered to be “natural skin supplements.” The combination of herbal ingredients with healthful and rejuvenating cactus juice products offers the health advantages of natural vitamins and herbs in an aromatic, pleasing and healthful skin application.

Ginseng root also has beneficial physiological effects. It is believed to help regulate blood pressure and increase the body's resistance to adverse physical, chemical and biological influences. Ginseng root can stimulate physical and mental activity and protect against the adverse effects of mental and physical stress. It may also improve concentration and stimulate brain cells. Ginseng root may be considered to be an herbal stimulant.

Presently preferred embodiments of the present invention may take the form of, for example and not by way of limitation, skin masques, skin creams, skin lotions, soaps, shampoos, facial cleansers, gels, exfoliant creams, hair conditioners and toothpastes.

The following tables further illustrate example formulas listing the ingredients currently used in preferred embodiments of the present invention. Ingredients listed in these tables are given by weight percentage of the total mixture.

### EXAMPLE 1

Replenishing Masque for Dry Skin

| | |
|---|---|
| Cactus (Cereus Grandiflorus) Fruit | 5% |
| Cactus (Cereus Grandiflorus) | 3% |
| Ginseng (Panax Ginseng) Berry | 3% |
| Water | 28.9% |
| SD Alcohol 40B | 10% |
| Glycerin | 7% |
| Hybrid Sunflower (Helianthus Annuus) Oil | 6% |
| Polyacrylamide | 5% |
| C13-14 Isoparaffin | 5% |
| Laureth-7 | 5% |
| Cyclomethicone | 5% |
| Grape (Vitis Vinifera) Seed Extract | 3% |
| Ginseng (Panax Ginseng) Root | 2% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Flowery Knotweed (Polygonum Aviculare) | 1% |
| Hibiscus Sabdariff | 1% |
| Mulberry (Morus Alba) Leaf | 1% |
| Riboflavin (Vitamin B2) | 1% |
| Tocopheryl Acetate (Vitamin E Acetate) | 1% |
| Niacin (Vitamin B3) | 1% |
| Pantothenic Acid (Provitamin B5) | 1% |
| Disodium Edta | 0.1% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 2

Intensive Night Repair

| | |
|---|---|
| Cactus (Cereus Grandiflorus) Fruit | 4% |
| Water | 33.9% |
| Shea Butter (Butyrospermum Parkii) | 4.5% |
| Glycerin | 4.5% |
| Cyclomethicone | 4.5% |
| Isopropyl Palmitate | 4% |
| Glyceryl Stearate | 4% |
| Stearic Acid | 4% |
| Sodium Behenoyl Lactylate | 4% |
| Ginseng (Panax Ginseng) Berry | 4% |
| Grape (Vitis Vinifera) Seed Extract | 4% |
| Ginseng (Panax Ginseng) Root | 4% |
| Cactus (Cereus Grandiflorus) | 4% |
| Avocado (Persea Gratissima) | 2% |
| Cucumber (Cucumis Sativus) | 2% |
| Jasmine (Jasminum Officinale) | 2% |
| Orange (Citrus Aurantium Dulcis) Peel | 2% |
| Flowery Knotweed (Polygonum Aviculare) | 1% |
| Hibiscus Sabdariff | 1% |
| Mulberry (Morus Alba) Leaf | 1% |
| Pyridoxine (Vitamin B6) | 1% |
| Riboflavin (Vitamin B2) | 1% |
| Tocopheryl Acetate (Vitamin E Acetate) | 1% |
| Pantothenic Acid (Provitamin B5) | 1% |
| Xanthan Gum | 0.2% |
| Carbomer | 0.2% |
| Disodium Edta | 0.2% |
| Phenoxyethanol | 0.2% |
| Methylparaben | 0.2% |
| Butylparaben | 0.2% |
| Ethylparaben | 0.2% |
| Propylparaben | 0.2% |
| Triethanolamine | 0.2% |

### EXAMPLE 3

Revitalizing Facial Cleanser

| | |
|---|---|
| Cactus (Cereus Grandiflorus) Fruit | 3% |
| Cactus (Cereus Grandiflorus) | 3% |
| Water | 54.5% |
| Sodium Cocoyl Isethionate | 5% |
| Sodium Methyl Cocoyl Taurate | 4% |
| Peg-8 | 4% |
| Octyldodecyl Benzoate | 3.5% |
| Myristic Acid | 3% |
| Glyceryl Stearate SE | 3% |
| Ginseng (Panax Ginseng) Root | 3% |
| Ginseng (Panax Ginseng) Berry | 3% |
| Chrysanthemum Coccineum | 1% |
| Cucumber (Cucumis Sativus) | 1% |
| Sage (Salvia Officinalis) | 1% |
| Grapefruit (Citrus Grandis) Peel | 1% |
| Kiwi (Actinidia Chinensis) | 1% |
| Lemon (Citrus Medica LI) | 1% |
| Algae | 1% |
| White Water Lily (Nymphaea Alba) | 1% |
| Niacin (Vitamin B3) | 1% |
| Tocopheryl Acetate (Vitamin E Acetate) | 1% |
| Butylene Glycol | 0.2% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 4

All-Day Hydrating Nourisher

| | |
|---|---|
| Cactus (Cereus Grandiflorus) Fruit | 3% |
| Cactus (Cereus Grandiflorus) | 3% |
| Water | 53% |
| Shea Butter (Butyrospermum Parkii) | 3.5% |
| Glycerin | 3.5% |
| Cyclomethicone | 3.5% |
| Glyceryl Stearate | 3% |
| Stearic Acid | 3% |
| Sodium Behenoyl Lactylate | 3% |
| Ginseng (Panax Ginseng) Berry | 3% |
| Grape (Vitis Vinifera) Seed Extract | 3% |
| Ginseng (Panax Ginseng) Root | 3% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Flowery Knotweed (Polygonum Aviculare) | 1% |
| Hibiscus Sabdariff | 1% |
| Mulberry (Morus Alba) Leaf | 1% |
| Pyridoxine (Vitamin B6) | 1% |
| Riboflavin (Vitamin B2) | 1% |
| Tocopheryl Acetate (Vitamin E Acetate) | 1% |
| Pantothenic Acid (Provitamin B5) | 1% |
| Xanthan Gum | 0.2% |
| Carbomer | 0.2% |
| Disodium Edta | 0.1% |
| Phenoxyethanol | 0.2% |
| Methylparaben | 0.2% |
| Butylparaben | 0.2% |
| Ethylparaben | 0.2% |
| Propylparagen | 0.2% |
| Triethanolamine | 0.2% |

### EXAMPLE 5

Time Signature Pure Cactus Ginseng Masque

| | |
|---|---|
| Cactus (Cereus Grandiflorus) Fruit Extract | 27.9% |
| Cactus (Cereus Grandiflorus) Extract | 15% |
| Ginseng (Panax Ginseng) Berry Extract | 20% |
| Ginseng (Panax Ginseng) Root Extract | 5% |
| SD Alcohol 40B | 3% |
| Glycerin | 3% |
| Hybrid Sunflower (Helianthus Annuus) Oil | 2% |
| Polyacrylamide | 2% |
| C13-14 Isoparaffin | 2% |
| Laureth-7 | 2% |
| Cyclomethicone | 2% |
| Avocado (Persea Gratissima) | 2% |
| Cucumber (Cucumis Sativus) | 2% |
| Jasmine (Jasminum Officinale) | 2% |
| Orange (Citrus Aurantium Dulcis) Peel | 2% |
| Flowery Knotweed (Polygonum Aviculare) | 1% |
| Hibiscus Sabdariff | 1% |
| Mulberry (Morus Alba) Leaf | 1% |
| Riboflavin (Vitamin B2) | 1% |
| Tocopheryl Acetate (Vitamin E Acetate) | 1% |
| Niacin (Vitamin B3) | 1% |
| Pantothenic Acid (Provitamin B5) | 1% |
| Disodium Edta | 0.1% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 6

Replenishing Masque for Normal Skin

| | |
|---|---|
| Cactus (Cereus Grandiflorus) Fruit | 5% |
| Cactus (Cereus Grandiflorus) | 3% |
| Water | 39.9% |
| Glycerin | 5% |
| Cyclomethicone | 5% |
| Polyacrylamide | 5% |
| C13-14 Isoparaffin | 5% |
| Laureth-7 | 5% |
| Aluminum Starch Octenylsuccinate | 5% |
| Hybrid Sunflower (Helianthus Annuus) Oil | 5% |
| Ginseng (Panax Ginseng) Berry | 2% |
| Grape (Vitis Vinifera) Seed Extract | 2% |
| Ginseng (Panax Ginseng) Root | 1% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Flowery Knotweed (Polygonum Aviculare) | 1% |
| Hibiscus Sabdariff | 1% |
| Mulberry (Morus Alba) Leaf | 1% |
| Riboflavin (Vitamin B2) | 1% |
| Tocopheryl Acetate (Vitamin E Acetate) | 1% |
| Niacin (Vitamin B3) | 1% |
| Pantothenic Acid (Provitamin B5) | 1% |
| Disodium Edta | 0.1% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 7

Deep Purifying Clay Masque

| | |
|---|---|
| Cactus (Cereus Grandiflorus) Fruit | 2.5% |
| Cactus (Cereus Grandiflorus) | 3% |
| Water | 57.5% |
| Kaolin | 7% |
| Glycerin | 5% |
| Glyceryl Stearate SE | 5% |
| Bentonite | 3% |
| Ginseng (Panax Ginseng) Root | 3% |
| Ginseng (Panax Ginseng) Berry | 1% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Flowery Knotweed (Polygonum Aviculare) | 1% |
| Hibiscus Sabdariff | 1% |
| Mulberry (Morus Alba) Leaf | 1% |
| Riboflavin (Vitamin B2) | 1% |
| Tocopheryl Acetate (Vitamin E Acetate) | 1% |
| Niacin (Vitamin B3) | 1% |
| Pantothenic Acid (Provitamin B5) | 1% |
| Magnesium Aluminum Silicate | 1% |
| Phenoxyethanol | 0.2% |
| Methylparaben | 0.2% |
| Ethylparaben | 0.2% |
| Propylparaben | 0.2% |
| Disodium Edta | 0.2% |

### EXAMPLE 8

Advanced Spot Control

| | |
|---|---|
| Cactus (Cereus Grandiflorus) Fruit | 5% |
| Cactus (Cereus Grandiflorus) | 1% |
| Water | 61.4% |
| SD Alcohol 40B | 7% |
| Glycerin | 5% |
| Ginseng (Panax Ginseng) Berry | 5% |
| Grape (Vitis Vinifera) Seed Extract | 2% |
| Ginseng (Panax Ginseng) Root | 2% |
| Chrysanthemum Coccineum | 1% |
| Cucumber (Cucumis Sativus) | 1% |
| Sage (Salvia Officinalis) | 1% |
| Grapefruit (Citrus Grandis) Peel | 1% |
| Kiwi (Actinidia Chinensis) | 1% |
| Lemon (Citrus Medica Limonum) | 1% |
| Orange (Citrus Aurantium Dulcis) Peel | 1% |
| Niacin (Vitamin B3) | 1% |
| Folic Acid | 1% |
| Tocopheryl Acetate (Vitamin E Acetate) | 1% |
| Triclosan | 1% |
| Carbomer | 0.3% |
| Triethanolamine | 0.3% |

### EXAMPLE 9

Intensive Day Defense

| ACTIVE INGREDIENTS | |
|---|---|
| Octyl Methoxycinnamate | 2% |
| Oxybenzone | 2% |
| Avobenzone | 2% |
| Phenylbenzimidazole Sulfonic Acid | 2% |
| OTHER INGREDIENTS | |
| Cactus (Cereus Grandiflorus) Fruit | 2% |
| Cactus (Cereus Grandiflorus) | 1% |
| Water | 45.5% |
| Aloe Barbadensis Gel | 4% |
| Glycerin | 4% |
| Octyl Stearate | 3% |
| C12-15 Alkyl Bezoate | 3% |
| Stearic Acid | 3% |
| Glyceryl Stearate | 3% |
| Isopropyl Palmitate | 2.5% |
| Octocrylene | 2% |
| Sodium Stearoyl Lactylate | 2% |
| Ginseng (Panax Ginseng) Berry | 2% |
| Grape (Vitis Vinifera) Seed Extract | 2% |
| Ginseng (Panax Ginseng) Root | 2% |
| Avocado (Persea Gratissima) | 1% |
| Cucumber (Cucumis Sativus) | 1% |
| Jasmine (Jasminum Officinale) | 1% |
| Pyridoxine (Vitamin B6) | 1% |
| Riboflavin (Vitamin B2) | 1% |
| Tocopheryl Acetate (Vitamin E Acetate) | 1% |
| Ascorbic Acid (Vitamin C) | 1% |
| Niacin (Vitamin B3) | 1% |
| Triethanolamine | 1% |
| Potassium Hydroxide | 1% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 10

Body Wash

| | |
|---|---|
| Cactus (Cereus Grandiflorus) Fruit | 4% |
| Cactus (Cereus Grandiflorus) | 4% |
| Water | 53% |
| Decyl Glucoside | 6% |
| Aloe Barbadensis Gel | 6% |
| Peg-120 Methyl Glucose Dioleate | 6% |
| Ammonium Laureth Sulfate | 5% |
| Disodium Cocoamphodiacetate | 4% |
| Grape (Vitis Vinifera) Seed Extract | 1% |
| Chrysanthemum Coccineum | 1% |
| Cucumber (Cucumis Sativus) Sage | 1% |
| Kiwi (Actinidia Chinensis) | 1% |
| Lemon (Citrus Medica Limonum) | 1% |
| Rose (Rosa Damascena) | 1% |
| White Water Lily (Nymphaea Alba) | 1% |
| Riboflavin (Vitamin B2) | 1% |
| Tocopheryl Acetate (Vitamin E Acetate) | 1% |
| Peg-7 Glyceryl Cocoate | 1% |
| Cocamidopropyl Betaine | 1% |
| Fragrance | 0.2% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 11

Hydrating Exfoliant

| | |
|---|---|
| Cactus (Cereus Grandiflorus) Fruit | 4% |
| Cactus (Cereus Grandiflorus) Stem | 3% |
| Water | 44.5% |
| Disodium Laureth Sulfosuccinate | 6% |
| Polyethylene | 5% |
| Glycerin | 5% |
| Cocamidopropyl Betaine | 4.5% |
| Peg-120 Methyl Glucose Dioleate | 4% |
| Triethanolamine | 4% |
| Ginseng (Panax Ginseng) Root | 3% |
| Grape (Vitis Vinifera) Seed Extract | 2% |
| Ginseng (Panax Ginseng) Berry | 2% |
| Chrysanthemum Coccineum | 1% |
| Cucumber (Cucumis Sativus) | 1% |
| Sage (Salvia Officinalis) | 1% |
| Grapefruit (Citrus Grandis) Peel | 1% |
| Kiwi (Actinidia Chinensis) | 1% |
| Lemon (Citrus Medica Limonum) | 1% |
| Rose (Rosa Damascena) | 1% |
| Algae | 1% |
| White Water Lily (Nymphaea Alba) | 1% |
| Niacin (Vitamin B3) | 1% |
| Tocopheryl Acetate (Vitamin E Acetate) | 1% |
| Polysorbate 20 | 1% |
| Benzophenone-4 | 0.2% |
| Phenoxyethanol | 0.2% |
| Methylparaben | 0.2% |
| Butylparaben | 0.2% |
| Ethylparaben | 0.2% |
| Propylparaben | 0.2% |
| Carbomer | 0.2% |
| Disodium Edta | 0.2% |

### EXAMPLE 12

Hair Conditioner

| | |
|---|---|
| Cactus (Cereus Grandiflorus) Fruit | 6% |
| Cactus (Cereus Grandiflorus) | 6% |
| Cactus (Cereus Grandiflorus) Stem | 2% |
| Water | 56% |
| Isopropyl Palmitate | 6% |
| Behentrimonium Methosulfate | 6% |
| Grape (Vitis Vinifera) Seed Extract | 3% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Chrysanthemum Coccineum | 1% |
| Pantothenic Acid (Provitamin B5) | 1% |
| Phytantriol | 1% |
| Folic Acid | 1% |
| Biotin (Vitamin H) | 1% |
| PG-Hydroxyethylcellulose Cocodimonium Chloride | 1% |
| Tridecyl Stearate | 1% |
| Neopentyl Glycol Disaprylate | 1% |
| Tridecyl Trimellitate | 1% |
| Silk Amino Acids | 1% |
| Phenoxyethanol | 0.2% |
| Methylparaben | 0.2% |
| Butylparaben | 0.2% |
| Ethylparaben | 0.2% |
| Propylparaben | 0.2% |

### EXAMPLE 13

All-Day Hydrating Nourisher for Oily Skin

| | |
|---|---|
| Cactus (Cereus Grandiflorus) Fruit | 4% |
| Cactus (Cereus Grandiflorus) | 2% |
| Water | 52.8% |
| Glycerin | 5% |
| Cyclomethicone | 5% |
| Polyacrylamide | 4% |
| C13-14 Isoparaffin | 4% |
| Laureth-7 | 4% |
| Ginseng (Panax Ginseng) Berry Root | 4% |
| Grape (Vitis Vinifera) Seed Extract | 2% |
| Ginseng (Panax Ginseng) | 2% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Flowery Knotweed (Polygonum Aviculare) | 1% |
| Hibiscus Sabdariff | 1% |
| Tocopheryl Acetate (Vitamin E Acetate) | 1% |
| Ascorbic Acid (Vitamin C) | 1% |
| Folic Acid | 1% |
| Acrylates Copolymer | 0.5% |
| Isopropyl Palmitate | 0.5% |
| Disodium Edta | 0.2% |
| Phenoxyethanol | 0.2% |
| Methylparaben | 0.2% |
| Butlyparaben | 0.2% |
| Ethylparaben | 0.2% |
| Propylparaben | 0.2% |
| Carbomer | 0.2% |
| Triethanolamine | 0.2% |

### EXAMPLE 14

Time Signature, Cactus Ginseng Essence

| | |
|---|---|
| Cactus (Cereus Grandiflorus) Fruit Extract | 45.3% |
| Cactus (Cereus Grandiflorus) Extract | 5% |
| Ginseng (Panax Ginseng) Berry Extract | 15% |
| Ginseng (Panax Ginseng) Root Extract | 3% |
| Grape (Vitis Vinifera) Seed Extract | 3% |
| Shea Butter (Butyrospermum Parkii) | 3% |
| Glycerin | 3% |
| Cyclomethicone | 3% |
| Isopropyl Palmitate | 2% |
| Glyceryl Stearate | 2% |
| Stearic Acid | 2% |
| Sodium Behenoyl Lactylate | 1% |
| Avocado (Persea Gratissima) | 1% |
| Cucumber (Cucumis Sativus) | 1% |
| Jasmine (Jasminum Officinale) | 1% |
| Orange (Citrus Aurantium Dulcis) Peel | 1% |
| Flowery Knotweed (Polygonum Aviculare) | 1% |
| Hibiscus Sabdariff | 1% |
| Mulberry (Morus Alba) Leaf | 1% |
| Pyridoxine (vitamin B6) | 1% |
| Riboflavin (Vitamin B2) | 1% |
| Tocopheryl Acetate (Vitamin E Acetate) | 1% |
| Pantothenic Acid (Provitamin B5) | 1% |
| Xanthan Gum | 0.3% |
| Carbomer | 0.3% |
| Disodium Edta | 0.1% |
| Phenoxyethanol | 0.2% |
| Methylparaben | 0.2% |
| Butlyparaben | 0.2% |
| Ethylparaben | 0.2% |
| Propylparaben | 0.2% |
| Triethanolamine | 0.2% |

### EXAMPLE 15

Ingredients for Happy Smile Tooth Paste:

| | |
|---|---|
| Cactus Fruit | 4% |
| Stevia | 25% |
| Deionized Water | 23.6% |
| Hydrated Silica | 20% |
| Sorbitol | 18% |
| Ginseng | 4% |
| Sodium Lauroyl Sarcosinate | 1.5% |
| Flavor | 1% |
| Peg-6 | 0.8% |
| Tetrasodium Pyrophosphate | 0.5% |
| Cellulose gum | 0.5% |
| Sodium Benzoate | 0.5% |
| Triclosan | 0.3% |
| Hydrogen Peroxide | 0.3% of 35% actives |

### EXAMPLE 16

Bar Soap Formula:

| | |
|---|---|
| Cactus Fruit Extract | 4% |
| Sodium Palmate | 55% |
| Sodium Cocoate | 16% |
| Water | 14.9% |
| Glycerin | 3% |
| Ginseng Berry Extract | 2% |
| Sorbitol | 2% |
| Spearic Acid | 2% |
| PPG-10 Methyl Glucose Ether | 1% |
| Pentasodium Pentetate | 0.05% |
| Tetrasodium Etidronate | 0.05% |

1. A skin care product comprising:
a cactus fruit extract; and,
a natural skin supplement.

2. The skin care product of claim 1 wherein said natural skin supplement is an herb.

3. The skin care product of claim 1 wherein said natural skin supplement is an herbal stimulant.

4. The skin care product of claim 1 wherein said natural skin supplement is ginseng root.

5. The skin care product of claim 1 further comprising ginseng berry extract.

6. The skin care product of claim 1 further comprising a skin masque composition.

7. The skin care product of claim 1 further comprising a facial cleanser composition.

8. The skin care product of claim 1 further comprising a body wash composition.

9. The skin care product of claim 1 further comprising a bar soap composition.

10. The skin care product of claim 1 further comprising a shampoo composition.

11. The skin care product of claim 1 further comprising a hair conditioner composition.

11. The skin care product of claim 1 further comprising a hair conditioner composition.

12. The skin care product of claim 1 further comprising a toothpaste composition.

13. A topical skin application comprising:
a cactus fruit extract; and,
a ginseng berry extract.

14. The topical skin application of claim 13 wherein said cactus fruit extract is taken from the Cereus Grandiflorus cactus.

15. The topical skin application of claim 13 wherein said cactus fruit extract is taken from the Opuntia cactus.

16. The topical skin application of claim 13 wherein said cactus fruit extract delivers vitamins to the skin upon application thereto.

17. The topical skin application of claim 13 wherein said cactus fruit extract moisturizes the skin upon application thereto.

18. The topical skin application of claim 13 wherein said cactus fruit extract enhances skin re-moisturizing.

19. The topical skin application of claim 13 wherein said cactus fruit extract promotes healing of wounds on the skin.

20. The topical skin application of claim 13 wherein said botanical ingredient is an herb.

21. The topical skin application of claim 13 wherein said botanical ingredient is ginseng root.

22. The topical skin application of claim 13 further comprising an ingredient selected from the group consisting of ginseng root, grape seed extract, avocado, cucumber, jasmine, orange peel, flowery knotweed, hibiscus sabdariff, mulberry leaf, chrysanthemum coccineum, sage, grapefruit peel, kiwi, lemon, algae, and white water lily.

23. The topical skin application of claim 13 wherein said application is a skin cream.

24. The topical skin application of claim 13 wherein said application is a hair care product.

25. A dental care product comprising:
a cactus fruit extract.

26. The dental care product of claim 25 further comprising ginseng berry.

27. A body treatment composition comprising:
a cactus fruit extract which, when applied to the skin, delivers vitamins to the skin, moisturizes the skin, and promotes healing of wounds on the skin; and,
a botanical ingredient.

28. The body treatment composition of claim 27 further comprising ginseng berry extract.
